(19) United States
(12) Patent Application Publication
(10) Pub. No.: US 2016/0262597 A1
(43) Pub. Date: Sep. 15, 2016

(54) PORTABLE ENDOSCOPIC SYSTEM

(71) Applicant: HYUNJU IN-TECH CO. LTD, Guro-gu, Seoul (KR)

(72) Inventors: Alexey DANCHINYU, Seoul (KR); Gyeong Ae SONG, Seoul (KR)

(21) Appl. No.: 14/403,578

(22) PCT Filed: Aug. 8, 2014
(86) PCT No.: PCT/KR2014/007375

§ 371 Date: Nov. 25, 2014

(30) Foreign Application Priority Data
Aug. 23, 2013 (KR) 10-2013-0100121

(51) Int. Cl. A61B 1/00; A61B 1/002; A61B 18/24; A61B 1/07; A61B 1/06; A61B 1/045

(52) U.S. Cl. CPC A61B 1/00108; A61B 1/0684; A61B 1/045; A61B 18/24; A61B 1/07; A61B 1/00066; A61B 1/00105; A61B 1/002; A61B 1/00142; A61B 1/0646; A61B 1/0638; A61B 2018/00982

(57) ABSTRACT

The present invention provides a portable endoscopic system, and more particularly a portable endoscopic system including a handle and a probe detachably coupled to the handle, the endoscopic system including: an illumination unit having at least one LED as a light source to emit a light forward; an imaging unit for imaging the forward light emitted from the illumination unit and displaying the image on an external image display; and a laser unit for performing medical treatment of an affected area displayed on the external image display, wherein the illumination unit, the imaging unit and the laser unit are integrally mounted in the handle.

### TECHNICAL FIELD

The present invention relates to a portable endoscopic system, and more particularly to a portable endoscopic system including a handle and a probe detachably coupled to the handle, the endoscopic system including: an illumination unit having at least one LED as a light source to emit a light forward; an imaging unit for imaging the forward light emitted from the illumination unit and displaying the image on an external image display; and a laser unit for performing a medical treatment of an affected area displayed on the external image display, wherein the illumination unit, the imaging unit and the laser unit are integrally mounted in the handle.

### BACKGROUND ART

Generally, an endoscopic system is a novel system that simplifies labor-intensive surgery in modern medical science. Such an endoscopic system is presently extensively applied to various surgical operations and is critical, for example, in ophthalmology. Such an endoscopic system may be critically applied to surgery for glaucoma, Endoscopic cyclophotocoagulation (ECP), and Implantation of intraocular lenses (IOL) without capsular support (Scleral fixation).

Glaucoma, which is a disease of optic nerve mainly caused by an intraocular pressure (IOP), is very difficult to recover from once it occurs. Primary open-angle glaucoma (POAG) is the most common among various kinds of glaucomas. When it is decided to commence a surgery for glaucoma, two approaches may be chiefly considered. The first approach that is predominantly utilized is to increase the amount of aqueous humor. This approach is referred to as a filtering surgery. However, one of key problems of filtering surgery is overfiltration. The second approach is to decrease generation of aqueous humor so as to decrease intraocular pressure. However, this involves a procedure that incurs the breakage of ciliary body. For this reason, surgery has to be executed through a sclera after cyclocryotherapy or photocoagulation of a ciliary body by a laser.

Because an operator cannot observe the objective tissue in execution of this surgery, neighboring tissue adjacent to the objective tissue may be damaged. Consequently, the surgical procedure is relatively complicated, and may involve incurrence of pain, a decrease in vision, inflammation, hypotony and phthisis bulbi.

An endoscopic cyclophotocoagulation (ECP) apparatus includes a semiconductor laser source, an endoscope and a xenon source all of which are provided in the same probe. The apparatus may be divided into two main units, one of which is a microprobe of 20 Ga and a camera and the other of which is a station in which a light source and a processor connected to a hybrid photoelectric cable are incorporated.

The apparatus however has disadvantages as follows.

1) Because an optical fiber bundle that is limited in the number of pixels required for transmission of image is used, the resolution of an imaging device is low.

2) Because means for emitting a laser beam from a probe is fixedly coupled to the probe, it is difficult to focus on the objective tissue. More specifically, because there is no additional device for controlling a focus or an emission direction of a laser beam, not only the objective tissue region but also the neighboring tissue may be damaged. In other words, because there is no means for focusing a laser beam, an output power required for photocoagulation must be increased and healthy tissue may be unintentionally removed.

3) Bulky imaging system and light source box have to be used. Consequently, a certain restriction in selection of operation position is involved in the case of indoor use. Since such an apparatus is occasionally installed and used in a permanent or near-permanent manner, a site for accommodating the apparatus has to be especially considered and adopted.

4) Although an endoscope utilizing a white light is a fundamental device for common examination of a tissue, there is a limitation in that the endoscope can allow observation only by a white light reflected from a mucous surface. Accordingly, it is difficult to achieve thorough observation of an objective tissue, and a critical shape of an objective tissue region observed during a diagnosis or surgical procedure may be overlooked. In order to more dearly observe a specific histological shape, observation by the white light has to be assisted by provision of an auxiliary device such as a narrow band imaging (NBI) device or a fluorescence imaging device.

Narrow band imaging enables a specific shape of a mucous surface to be more clearly observed by the use of blue and green lights having specific wavelengths. In general, narrow band imaging electromagnetically activates a special filter of a light source for an endoscope so that peripheral lights such as a blue light having a wavelength of 440-460 nm and a green light having a wavelength of 540-560 nm can be used. Since the optical absorption peak occurs in these wavelength ranges, blood vessels appear to be very dark, thus relatively improving visibility of a tissue such as a mucous membrane and improving the ability to discriminate other surfaces.

U.S. Pat. No. 7,063,663 discloses an endoscopic system including an endoscope and an illumination assembly. For the endoscopic system, a series of LEDs are incorporated in the end of the endoscope. Although the design of the system is suitable for solving a problem of space limitation caused by separate provision of a light source box and a light guide, the design is still bulky and complicated, and is thus not suitable to be portable.

### DISCLOSURE

### Technical Problem

Accordingly, the present invention has been made keeping in mind the above problems occurring in the prior art, and an object of the present invention is to provide a portable endoscopic system in which all components required for laser photocoagulation and accurate video guidance are integrally incorporated.

The system provides a portable integral package in which an imaging system, a light illumination system, a tissue-removing system allowing operation and focusing, and a replaceable probe head are integrally incorporated.

Another object of the present invention is to provide a portable endoscopic system that allows control of laser focusing so as to minimize damage of neighboring tissues in execution of laser photocoagulation.

A further object of the present invention is to provide a portable endoscopic system that, thanks to adoption of a rod lens, assures a higher resolution than that in the case of adoption of optical fibers and enables easy control of emission direction and emission region of a laser by means of mechanical displacements of the lens and a mirror.

Yet another object of the present invention is to provide a portable endoscopic system that realizes a higher contrast and clearly specifies an object to be treated by introduction of a narrow band imaging technology.

### Technical Solution

In order to accomplish the above objects, the present invention provides a portable endoscopic system including a handle and a probe detachably coupled to the handle, the endoscopic system including: an illumination unit having at least one LED as a light source to emit a light forward; an imaging unit for imaging the forward light emitted from the illumination unit and displaying the image on an external image display; and a laser unit for performing medical treatment of an affected area displayed on the external image display, wherein the illumination unit, the imaging unit and the laser unit are integrally mounted in the handle.

The illumination unit may include: a branched optical fiber bundle for concentrating the light emitted from the LED and directing the concentrated light forward; and a collimating lens for collimating the light emitted from the branched optical fiber bundle.

The imaging unit may include an imaging CCD or CMOS sensor composed of a PCB and a sensor-driving circuit disposed on the PCB, and a converter that is disposed in front of the sensor so as to control a focus of an image by controlling displacement of an imaging lens in accordance with a shooting position, the imaging CCD or CMOS sensor and the converter being sequentially arranged in a housing longitudinally formed in the handle.

The converter may include: an imaging bracket that is coupled to the imaging lens and is longitudinally movable; an imaging knob that is rotatably mounted on an external surface of the handle to control the focus of the imaging lens; and an imaging rod that is disposed between the imaging bracket and the imaging knob so as to transmit a rotational force of the imaging knob to the imaging bracket thus converting the rotational movement into a reciprocating movement.

The laser unit may include a controller that is provided on the handle to control an emission direction and focus of a laser.

The controller may include: a first controller that displaces one of a pair of laser lenses through which the laser passes to control the focus of the laser; and a second controller for controlling an emission direction of the laser that has passed through the first controller.

The first controller may include: a laser bracket that is connected at one end thereof to one of the pair of laser lenses and is provided at the other end thereof with a toothed section; a laser gear that engages with the laser bracket so as to move the laser bracket in a longitudinal direction of the handle during rotation thereof; and a laser dial that is partially exposed to the outside through the handle and engages with the laser gear, the laser dial rotating the laser gear to longitudinally move the laser bracket during rotation thereof.

The second controller may include: a pair of reflecting members composed of a first reflector and a cube prism for diverting an emission direction of a laser that has passed through the first controller; and a directional control button provided on the handle and connected to the first controller of the pair of reflecting members so as to control an angle of the first reflector and thus an emission direction of a laser.

The directional control button may have a panel shape and is pressed in all directions, the directional control button including a concave recess formed on an exposed surface thereof, and a pressed position of the directional control button is restored to a rest position by an elastic element elastically supporting an internal surface of the control button.

The elastic element may be at least one leaf spring or coil spring supporting a circumferential region of the directional control button.

The handle may include a cube prism that integrates a channel of the imaging unit and a channel of the laser unit and makes the two channels concentric at an end of an endoscope.

The handle may further include a glass window for preventing contamination through a side thereof coupled to the probe.

The probe may be provided with a rigid rod lens.

The illumination unit may be positioned at an upper level of the handle, the imaging unit may be positioned at an intermediate level of the handle and the laser unit may be positioned at a lower level of the handle, and wherein the illumination unit, the imaging unit and the laser unit may be integrated with each other in the handle.

The imaging unit may further include an image effect unit for improving visibility and legibility of a forward image or tissue.

The image effect unit may include: a filter part that is disposed between the cube prism and the imaging lens to offer imaging of two or more visual effects of a forward image; a filter knob that is rotatably installed on an external surface of the housing; and a filter rod that is installed between the filter part and the filter knob to transmit a rotational force of the filter knob to the filter part for conversion of the forward image.

The filter part may include a filter body coupled to the filter rod and having at least one filter radially disposed, and wherein the at least one filter includes two or more filters that are radially provided on the filter body to offer different visual effects.

The at least one LED may include a plurality of LEDs that emit a white light and a blue or green light, respectively.

### Advantageous Effects

According to the present invention, an endoscopic system in which all components required for laser photocoagulation and accurate video guidance are integrally incorporated is provided, and thus it assures convenience in use and expanded area of use.

Furthermore, since the focus of laser can be mechanically controlled during laser photocoagulation, flexible application of the laser can be allowed in accordance with a position of an objective tissue, thus minimizing damage of a neighboring tissue adjacent to the objective tissue during an operation.

In addition, thanks to adoption of a rod lens having a relatively high resolution, a higher resolution than that in the case of adoption of optical fibers is assured and control of the laser emission direction and emission region by means of mechanical displacements of the lens and a mirror is facilitated.

Furthermore, a higher contrast is realized and an object to be treated is clearly specified by introduction of narrow band imaging technology.

### DESCRIPTION OF DRAWINGS

FIG. 1 shows a front view, a side view and a rear view of an endoscopic system according to an embodiment of the present invention;

FIG. 2 is a cross-sectional view of a handle and a probe according to the embodiment of the present invention, which are separated from each other;

FIG. 3 is a fragmentary cross-sectional view of an endoscope according to the embodiment of the present invention, which includes a detail view of an illumination system;

FIG. 4 shows two types of branched optical fiber bundles, that is, a usual optical fiber bundle and an optical fiber bundle having a tapered end;

FIG. 5 shows a laser beam steering and focusing mechanism according to the embodiment of the present invention;

FIG. 6 shows an emission direction of a laser beam, which is changed by movement of a directional control button;

FIG. 7 is a cross-sectional view showing an image effect unit; and

FIG. 8 shows various parameters of a replaceable probe of the endoscope.

### BEST MODE

Hereinafter, a preferred embodiment of the present invention will be described in more detail with reference to the accompanying drawings.

A portable endoscopic system according to the present invention has discriminative effects as compared to conventional technology.

1) An endoscope according to the present invention includes a laser photocoagulation (tissue removal) system that is mounted concentrically with an image channel. Consequently, this enables realization of direct and consecutive image processing and exact photocoagulation and makes the overall size of the system very compact.

2) A laser photocoagulation system according to the present invention includes means for concentrating a laser beam and a controller, thus enabling removal of the objective tissue to be treated while minimizing damage occurring to a neighboring tissue that is not the objective tissue.

3) By incorporation of a narrow band imaging system, a higher contrast is realized thus enabling a precise medical operation.

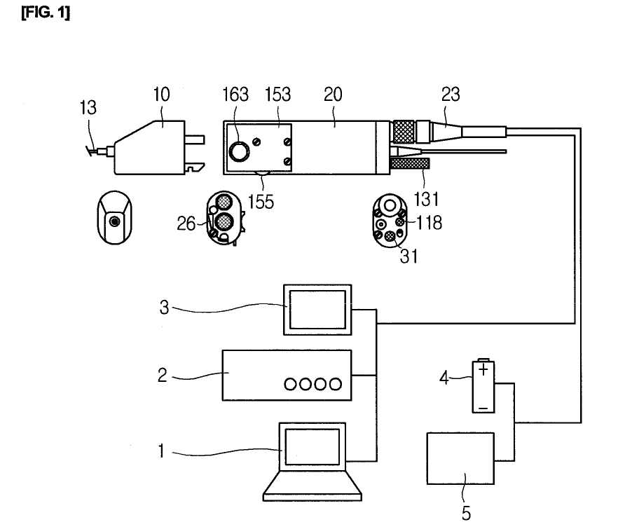

As illustrated in FIG. 1, the endoscopic system according to the present invention is composed of two main components, one of which is a handle 20 that includes therein an illumination unit 110, an imaging unit 120 and a laser unit 150 and includes thereon a laser beam focusing controller 153. A direction of radiation of a laser is controlled using a direction control button 167, and a focus of the laser is controlled using a laser dial 157. The other component of the endoscopic system is a detachable probe 10. The detachable probe 10 is optionally used depending on optical properties and an imaging efficiency.

More specifically, the endoscopic system comprises the illumination unit 110 that functions to radiate a light forwardly from an LED 113 serving as a light source, the imaging unit 120 that converts the forward light emitted from the illumination unit 110 into an image and displays the image on an external image display, and the laser unit 150 that performs removal or treatment of a body tissue displayed on the external image display 120. Particularly, the illumination unit 110, the imaging unit 120 and the laser unit 150 are integrally mounted in the handle 20, thus making the endoscopic system portable, light and compact. Particularly, the illumination unit 110, the imaging unit 120 and the laser unit 150 are each longitudinally arranged in the handle 20 such that the illumination unit 110 is disposed at an upper level of the handle 20, the imaging unit 120 is disposed at n intermediate level of the handle 20 and the laser unit 150 is disposed at a lower level of the handle 20, thus allowing integration of the illumination unit 110, the imaging unit 120 and the laser unit 150 in the handle 20.

Therefore, features of the present invention that make the endoscopic system small and light are realized through the arrangement of the components in the handle 20.

The probe 10 is connected to the handle 20 through a coupling device (not shown) that also allows the optical alignment between the handle 20 and the probe 10. A detachable lever 25 is provided on a lateral surface of the handle 20, so that the probe 10 can be separated from the handle 20 by pushing the detachable lever 25.

An endoscope is directly connected to a monitor 3 via a multi-pin connector 23, thus enabling a test object to be instantly observed. Furthermore, since the endoscopic system is constructed to be compact, lightweight and thus portable, it can be instantly used at any site. Since the endoscope is connected to an image processing unit 2 or a computer 1, minute image processing such as adjustment of contrast and improvement of white balance and color is possible.

Adjustment of a focal length due to replacement of the probe 10 can be fulfilled by manipulation of a laser dial 161 disposed on a rear surface of the endoscope. When the probe 10 including a rod lens 13 is used, a distance between an end of the probe 10 and a suspected area can be adjusted while the endoscope is fixed by means of the laser dial 161.

Consequently, when a laser is used in a small space, any tissues that are positioned at different distances from the end of the endoscope can be ablated without damage of neighboring tissues.

A switch 31 is used in order to connect an illumination source and a camera to an external power source. The power source is selected among an external battery pack of a voltage inverter.

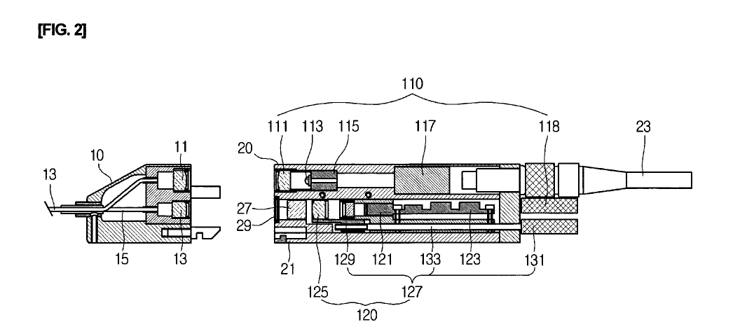

FIG. 2 is a cross-sectional view of an embodiment of the present invention in which the probe is separated from the handle. In the drawing, laser operating and focusing mechanisms are not illustrated.

The illumination unit 110 includes one or two LEDs 113 that are mounted on separated heat sinks 115, respectively. The two LEDs 113 are activated by means of a single LED driver 117.

The heat sinks 115 partially remove heat generated from the LEDs 113 and discharge the heat outside through a housing 21.

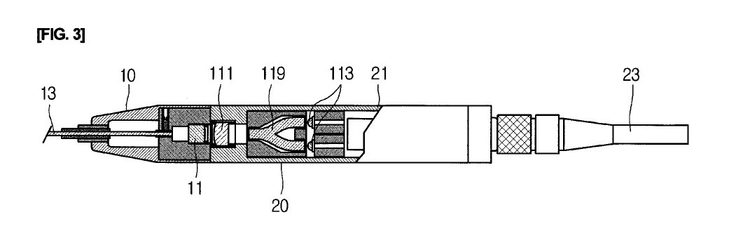

As illustrated in FIG. 3, the illumination unit 110 preferably comprises a branched optical fiber bundle 119 for concentrating lights emitted from the LEDs 113 and directing the concentrated lights forward, and a collimating lens 111 for collimating the lights emitted from the branched optical fiber bundle 119. The LEDs 113 are composed of two LEDs 113. The light beams emitted from the LEDs 113 are joined together at one point through the branched optical fiber bundle 119.

In this embodiment, the LEDs 113 are composed of two LEDs 113 one of which emits a white light and the other of which emits a blue or green light. The two LEDs 113 are selectively activated to provide a white light image or a narrow band image by means of a switch, as required. The LEDs 113 may be composed of more than two LEDs 113.

Alternatively, the two LEDs 113 may emit only a white light and the lens of the imaging unit may be provided with a filter, so as to allow a white light image and a narrow band image to be selectively observed by an operator, which will be described later. In other words, the case where the LEDs 113 that emit different lights are used without the filter and the case where the filter and the LEDs 113 that emit the same light are used belong to different embodiments.

Although the branched optical fiber bundle 119 may be replaced with a dichroic beam splitter, the branched optical bundle 119 is more advantageous because the dichroic beam splitter is more expensive.

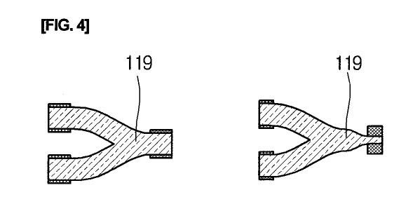

Another advantage of the branched optical fiber bundle 119 resides in the fact that the end of the branched optical fiber bundle can be tapered so as to have a reduced diameter, as shown in FIG. 4. When a diameter of the end is reduced owing to the tapered configuration, a spot of the illumination source can be reduced thus enhancing an efficiency of projection.

For example, when a diameter of the optical fiber bundle 119 is reduced to ½, the radiant intensity can increase up to four times. When the diameter of the optical fiber bundle 119 is reduced to ⅓, the radiant intensity can increase up to nine times. Thanks to the enhancement of the illumination efficiency, the power consumption of the LEDs 113 can also be reduced.

The emitted spectrum can be changed by replacing the white light of the LEDs 113 with a green light or a blue light. In order to make an emitted waveband narrower and to increase a signal-to-noise ratio at the time of imaging, a narrow band filter may be disposed between the LEDs 113 and the optical fiber bundle 119.

More specifically, a filter-exchanging lever 118 is used to convert a white light imaging mode (W) into a narrow band imaging mode (NB). Specifically, the filters include a colorless transparent filter and a color filter such as a green filter or a blue filter. The white light imaging and the narrow band imaging may be replaced by each other by disposing require filters on the LEDs 113 using the filter-exchanging lever 118.

When an object is observed in a narrow band spectrum, contrast is increased in observation of a body tissue or pathological diagnosis, thus enabling the body tissue to be distinctly discriminated from the neighboring area. At this point, colorant is previously injected into the neighboring area adjacent to the body tissue (Fluorescence endoscopy), or the phenomenon that colorant is more easily absorbed in the neighboring area of the body tissue at a specific spectrum band (narrow band imaging; NBI) is utilized. The filter-exchanging lever 12 mechanically rotates a filter exchanger positioned on the front face of the imaging unit 120. When it is necessary to obtain an image that exhibits an objective tissue to distinctly discriminate it from the neighboring tissue for the pathological observation, the contrast in the image has to be increased. Therefore, a body tissue can be observed at a NBW spectrum using a fluorescence endoscope, which is very advantageous in quantitative analysis of the tissue.

The narrow band imaging (NBI) is performed using the emitted light of a narrow band such as blue light having a wavelength of 440-460 nm or green light having a wavelength of 540-560 nm. Since the peak absorption of hemoglobin occurs at these wavelengths, a blood vessel appears very dark and thus visibility of hemoglobin is relatively improved. In addition, other surface structures can be more clearly discerned.

The light emitted from the end of the branched optical fiber bundle 119 is collimated through the collimating lens 111. The collimated light is concentrated to the end of the optical fiber bundle 119 of the detachable probe 10 through a focusing lens 11.

The imaging unit 120 includes an imaging CCD or CMOS sensor 121 composed of a PCB 123 and a sensor-driving circuit disposed on the PCB 123, and a converter 127 that is disposed in front of the sensor 121 so as to control the focus of an image by controlling displacement of an imaging lens 125 in accordance with a shooting position. It is preferable that these components are sequentially arranged in the housing 21 that is longitudinally formed along the handle 20. The imaging lens 125 that is positioned in front of the sensor 121 radiates an image to a surface of the sensor 121.

The converter 127 comprises an imaging bracket 129 that is coupled to the imaging lens 125 and is longitudinally movable, an imaging knob 131 that is rotatably mounted on an external surface of the handle 20 to control the focus of the imaging lens 125, and an imaging rod 133 that is disposed between the imaging bracket 129 and the imaging knob 131 so as to transmit a rotational force of the imaging knob to the imaging bracket 129 thus converting the rotational movement into a reciprocating movement.

A cube prism 27 aggregates two channels such as an image and a laser and makes the two channels concentric. Furthermore, the cube prism 27 serves as a beam splitter that reflects a laser reflected at a first reflector 165 (described later) and directs the laser to the rod lens 13. The cube prism 27 is composed of a pair of prism elements each having a right triangle section such that hypotenuses of cross-sections of the prism elements are in contact with each other thus defining a square prism in its entirety. In this regard, a laser is refracted at the interface defined between the hypotenuses and is guided to the rod lens 13.

A glass window 29 functions to prevent the components from being contaminated by extraneous substances and humidity. The imaging lens 13 collimates an imaging beam exiting from an image relay optical device 15 and transmits the imaging beam to the focusing lens 11.

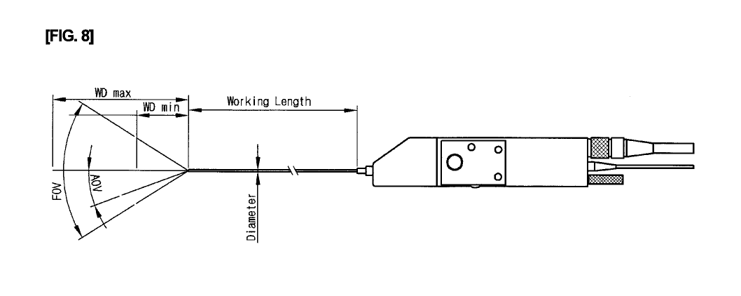

The imaging unit 120 further includes an image effect unit 140 that further improves visibility and legibility of a forward image or tissue by replacing the white light of the LEDs 113 with a green or blue light, as illustrated in FIG. 8.

The image effect unit 140 is intended to convert an image of white light into a narrow band image. In other words, when a test object is observed in the narrow band spectrum, contrast in pathological observation is increased thus enabling recognition of the type of tissue and allowing discrimination an object tissue from a neighboring area. Specifically, when a tissue in question is removed in a surgery operation, it allows the tissue in question to be discriminated from a normal tissue. At this point, the discrimination of the tissue is implemented by injecting colorant into a neighboring area of the tissue in question or causing the colorant to be more easily absorbed at a specific spectrum band.

The image effect unit 140 comprises a filter part 141 that is disposed between the cube prism 27 and the imaging lens 125 to offer imaging of two or more visual effects of a forward image, a filter knob 147 that is rotatably installed at a region adjacent to the imaging knob (not shown) disposed at the external surface of the housing 21, and a filter rod 149 that is installed between the filter part 141 and the filter knob 147 to transmit a rotational force of the filter knob 147 to the filter part 141 for conversion of the forward image.

The filter part 141 comprises at least a pair of filters 145 that is disposed between the cube prism 27 and the imaging lens 125 to convert a white light into a green or blue light thus offering two or more image effects, and a filter body 143 that is coupled to the filter rod 149 and is radially provided with the filters 145.

As illustrated in the drawing, the pair of filters 145 includes a first filter 145-1 for imaging the white light itself, and a second filter 145-2 for converting the white light into green or blue light and imaging the converted light. The filters 145 can be selectively applied through rotation of the filter knob 145. In this regard, it is preferable that the pair of filters 145 may be exchanged with each other in position depending on application.

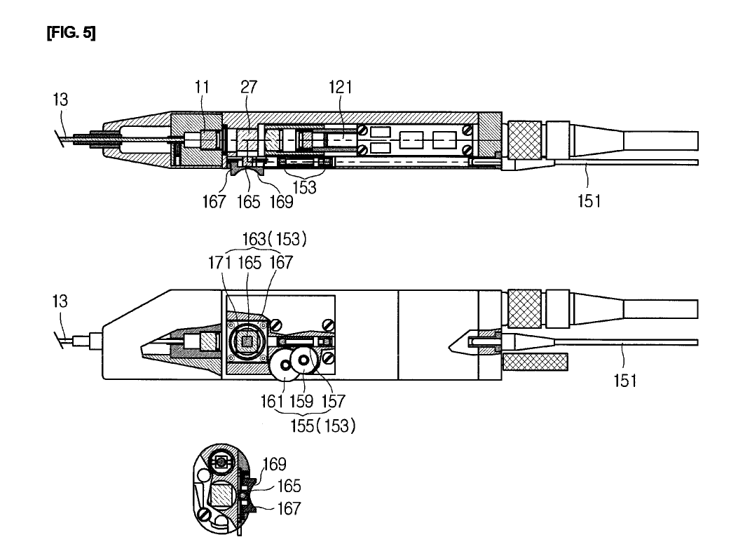

FIG. 5 shows a laser beam steering and focusing mechanism.

An external laser source composed of optical fibers is connected to the handle of the endoscope through a connector 151. In order to allow the collimated laser beam to be normally output, the collimated laser beam passes through two identical laser lenses 153 that are spaced apart from each other by a distance equal to the sum of focal lengths of the laser lenses.

The laser unit 150 constituting the channels includes the controller 153 that is provided on the handle 20 to control an emission direction and a focus of a laser.

The controller 153 comprises a first controller 155 that displaces one of the pair of laser lenses 153 through which the laser passes to control the focus of the laser, and a second controller 163 for controlling an emission direction of the laser that has passed through the first controller 155.

The first controller 155 comprises a laser bracket 157 that is connected at one end thereof to one of the pair of laser lens 153 and is provided at the other end thereof with a toothed section, a laser gear 159 that engages with the laser bracket 157 so as to move the laser bracket 157 in a longitudinal direction of the handle 20, and a laser dial 161 that is partially exposed to the outside through the handle 20 and engages with the laser gear 159, the laser dial 161 rotating the laser gear 159 to longitudinally move the laser bracket 159 during rotation thereof.

One of the pair of laser lenses 153 is connected to the first controller 155 and thus is movable longitudinally. The arrangement and the longitudinal movement of the lens enables a precise control of an angle of dispersion and thus a precise control of a position of the laser focus at the end of the rod lens 13.

The second controller 163 comprises a pair of reflecting members (a first reflector 165 and a cube prism 27) for diverting an emission direction of a laser that has passed through the first controller 155, and a directional control button 167 provided on the handle 20 so as to control an angle of the first reflector 165 and thus an emission direction of a laser.

The collimated beam is reflected by 90 degrees at the first reflector 165 connected to the directional control button 167. The first reflector 165 can move with the same degree of freedom as the directional control button 167, which has two degrees of freedom.

When the directional control button 167 is moved up and down or back and forth, an emission direction of a laser is correspondingly changed at the end of the endoscope. The directional control button 167 is elastically supported by an elastic element 171, so that the reference position of a laser beam at which the laser beam is emitted from the center of the rod lens 13 is maintained.

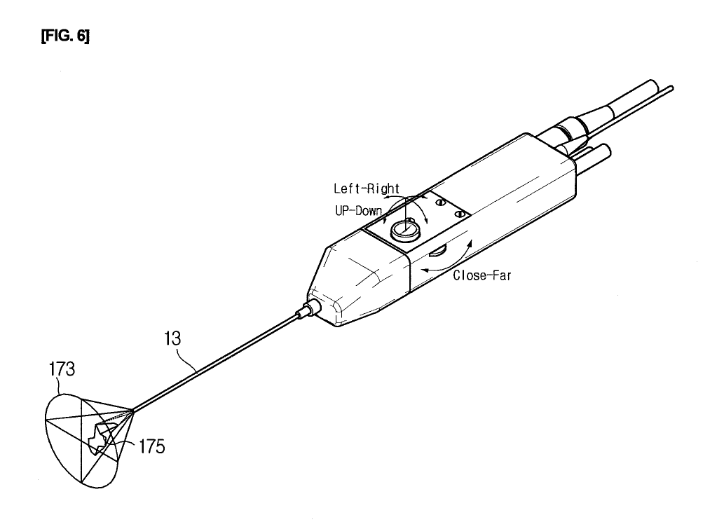

Since accurate focusing of the laser is obtained and the emission direction of the laser is controlled within the entire region of a field of view (173), the neighboring tissue is not damaged when an object in question is removed. FIG. 6 shows an emitted region of a laser emitted through the rod lens 13.

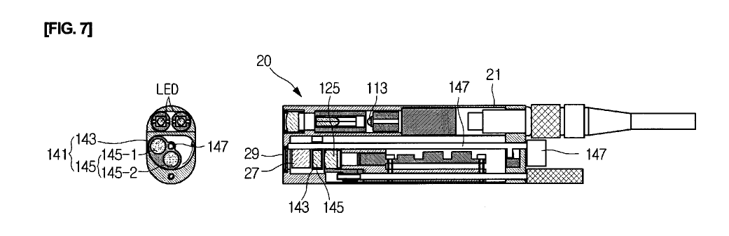

FIG. 7 shows various parameters that have an effect on the selection of replaceable endoscope probes.

A plurality of endoscope probes having various functions may be presented depending on length, diameter, a angle of view (AOV), field of view (FOV), degree of flexibility, working distance (VVD), diagnosis type or body region of a patient, and may be interchangeable with each other.

Thanks to the interchangeable feature, the endoscopic probe is very useful in a multipicture guided ENT surgery. Furthermore, a compact rigid probe can be applied to a field of Minimal Invasive Orthopedic Surgery (MIOS), more particularly to a field of arthroscopic surgery for a knee, a shoulder, a hand, a foot or a hip.

Furthermore, a subcompact rigid probe can be applied to fields of ophthalmic surgery and observance of ductus lactiferi. A flexible probe can be used in fields of urology and bronchoscopy for the purpose of diagnosis and medical treatment.

A flexible compact probe can be used in examination of an epidural space through a minimally invasive technique that is used in diagnosis and medical treatment of chronic back pain and radiculopathy.

### INDUSTRIAL APPLICABILITY

An endoscopic system is industrially used in examination and observation of regions that are difficult or dangerous to access. Furthermore, a replaceable probe may be manufactured into a reproducible or disposable product having a tool and/or a suction tube.

1. A portable endoscopic system including a handle and a probe detachably coupled to the handle, the endoscopic system comprising:
an illumination unit having at least one LED as a light source to emit a light forward;
an imaging unit for imaging the forward light emitted from the illumination unit and displaying the image on an external image display; and
a laser unit for performing a medical treatment of an affected area displayed on the external image display,
wherein the illumination unit, the imaging unit and the laser unit are integrally mounted in the handle.

2. The portable endoscopic system according to claim 1, wherein the illumination unit comprises:
a branched optical fiber bundle for concentrating the light emitted from the LED and directing the concentrated light forward; and
a collimating lens for collimating the light emitted from the branched optical fiber bundle.

3. The portable endoscopic system according to claim 1, wherein the imaging unit comprises an imaging CCD or CMOS sensor composed of a PCB and a sensor-driving circuit disposed on the PCB, and a converter that is disposed in front of the sensor so as to control a focus of an image by controlling displacement of an imaging lens in accordance with a shooting position, the imaging CCD or CMOS sensor and the converter being sequentially arranged in a housing longitudinally formed in the handle.

4. The portable endoscopic system according to claim 3, wherein the converter comprises:
an imaging bracket that is coupled to the imaging lens and is longitudinally movable;
an imaging knob that is rotatably mounted on an external surface of the handle to control the focus of the imaging lens; and
an imaging rod that is disposed between the imaging bracket and the imaging knob so as to transmit a rotational force of the imaging knob to the imaging bracket thus converting the rotational movement into a reciprocating movement.

5. The portable endoscopic system according to claim 1, wherein the laser unit comprises a controller that is provided on the handle to control an emission direction and focus of a laser.

6. The portable endoscopic system according to claim 5, wherein the controller comprises:
a first controller that displaces one of a pair of laser lenses through which the laser passes to control the focus of the laser; and
a second controller for controlling an emission direction of the laser that has passed through the first controller.

7. The portable endoscopic system according to claim 6, wherein the first controller comprises:
a laser bracket that is connected at one end thereof to one of the pair of laser lens and is provided at the other end thereof with a toothed section;
a laser gear that engages with the laser bracket so as to move the laser bracket in a longitudinal direction of the handle during rotation thereof; and
a laser dial that is partially exposed to the outside through the handle and engages with the laser gear, the laser dial rotating the laser gear to longitudinally move the laser bracket during rotation thereof.

8. The portable endoscopic system according to claim 6, wherein the second controller comprises:
a pair of reflecting members composed of a first reflector and a cube prism for diverting an emission direction of a laser that has passed through the first controller; and
a directional control button provided on the handle and connected to the first controller of the pair of reflecting members so as to control an angle of the first reflector and thus an emission direction of a laser.

9. The portable endoscopic system according to claim 8, wherein the directional control button has a panel shape and is pressed in all directions, the directional control button including a concave recess formed on an exposed surface thereof, and a pressed position of the directional control button is restored to a rest position by an elastic element elastically supporting an internal surface of the control button.

10. The portable endoscopic system according to claim 9, wherein the elastic element is at least one leaf spring or coil spring supporting a circumferential region of the directional control button.

11. The portable endoscopic system according to claim 1, wherein the handle comprises a cube prism that integrates a channel of the imaging unit and a channel of the laser unit and makes the two channels concentric at an end of an endoscope.

12. The portable endoscopic system according to claim 1, wherein the handle further comprises a glass window for preventing contamination through a side thereof coupled to the probe.

13. The portable endoscopic system according to claim 1, wherein the probe is provided with a rigid rod lens.

14. The portable endoscopic system according to claim 1, wherein the illumination unit is positioned at an upper level of the handle, the imaging unit is positioned at an intermediate level of the handle and the laser unit is positioned at a lower level of the handle, and wherein the illumination unit, the imaging unit and the laser unit are integrated with each other in the handle.

15. The portable endoscopic system according to claim 3, wherein the imaging unit further comprises an image effect unit for improving visibility and legibility of a forward image or tissue.

16. The portable endoscopic system according to claim 15, wherein the image effect unit comprises:
a filter part that is disposed between the cube prism and the imaging lens to offer imaging of two or more visual effects of a forward image;
a filter knob that is rotatably installed on an external surface of the housing; and
a filter rod that is installed between the filter part and the filter knob to transmit a rotational force of the filter knob to the filter part for conversion of the forward image.

17. The portable endoscopic system according to claim 16, wherein the filter part comprises a filter body coupled to the filter rod and having at least one filter radially disposed, and wherein the at least one filter includes two or more filters that are radially provided on the filter body to offer different visual effects.

18. The portable endoscopic system according to claim 1, wherein the at least one LED includes a plurality of LEDs that emit a white light and a blue or green light, respectively.
